(19) United States
(12) Patent Application Publication
(10) Pub. No.: US 2023/0228946 A1
(43) Pub. Date: Jul. 20, 2023

(54) SYSTEM LEAD CONNECTOR FOR PRESSURE GUIDEWIRE

(71) Applicant: Phyzhon Health Inc., Folsom, CA (US)

(72) Inventors: Michael J. Eberle, Fair Oaks, CA (US); Diana Margaret Tasker, Fair Oaks, CA (US); Howard Neil Rourke, Sacramento, CA (US); Bryan Foronda, Sacramento, CA (US)

(21) Appl. No.: 18/069,671

(22) Filed: Dec. 21, 2022

Related U.S. Application Data
(60) Provisional application No. 63/263,984, filed on Jan. 19, 2022.

(51) Int. Cl. G02B 6/36; G02B 6/38

(52) U.S. Cl. CPC G02B 6/3616; G02B 6/3897

(57) ABSTRACT

A medical device optical connector lead for coupling with a guidewire including optical fiber is described herein. The connector can include or use a housing defining an aperture, an optical receptacle disposed within the housing, the optical receptacle configured to receive an exposed optical fiber end of the guidewire extending through the aperture, and a chuck configured to clamp around the guidewire. The chuck can be slidable within the housing between a first chuck position wherein the chuck is positioned closer to the optical receptacle than to the aperture and a second chuck position wherein the chuck is positioned closer to the aperture than to the optical receptacle. An actuator can laterally move the chuck between the first position and the second position and concurrently tighten or loosen the chuck.

### CROSS REFERENCE TO RELATED APPLICATION

\ This patent application claims the benefit of U.S. Provisional Pat. Application No. 63/263,984, filed Jan. 19, 2022, entitled “SYSTEM LEAD CONNECTOR FOR PRESSURE MEASUREMENT GUIDEWIRE”, which is incorporated by reference herein in its entirety.

### TECHNICAL FIELD

This document pertains generally to pressure sensing devices, imaging devices, and methods and in particular to pressure sensing devices, imaging devices, and methods using optical elements and techniques.

### BACKGROUND

In a medical procedure a clinician can take a pressure measurement from within a body lumen of a patient, such as an artery or vein. For example, a pressure guidewire can be used to measure the pressure distal to a lesion, such as in the coronary vasculature. The pressure guidewire can include or use transducing elements or optical pressure elements. A pressure guidewire can be removably couplable to a console, e.g., an optical signal analyzer or conditioner, via an optical fiber. It can be desirable during a procedure to have the ability to reliably couple and uncouple the guidewire recurrently to the signal analyzer, such as to enable post stenting assessment or multi-vessel assessment.

### SUMMARY

The present inventors have recognized, among other things, devices, and techniques to help perform reliable, recurrent connections between a pressure guidewire and a console during a procedure. An optical system lead connector can be used such as to relay an optical signal from a distal end of a pressure guidewire to a console. The optical system lead connector can removably couple with a proximal end of the guidewire such as to create an optical connection with the optical fiber contained in the guidewire at an optical interface of the system lead connector. The guidewire can be recurrently reconnected several times during a procedure at the optical interface, potentially subjecting the proximal end of the guidewire to elements in the sterile field. Further, each of these recurrent optical connections between the proximal end of the guidewire and the optical interface of the optical system lead connector must be accurately and precisely aligned and contacted to achieve a suitable connection.

In one approach to recurrent guidewire connection, an optical system lead connector can include or use an optical fiber interface. For example, the interface can be a handle or a shaft, the interface sized and shaped to help the clinician hold or manipulate the guidewire. The optical fiber interface can include a mechanism, such as a chuck, for gripping the guidewire once it has been inserted into the interface. The mechanism can be actuated by a dial, knob, or other mechanism arranged such that when turned, members of the interface clamp on the guidewire and prevent it from being removed during the procedure. A problem with this single-action mechanism is that it may not necessarily enforce a reliable optical connection along with gripping the guidewire and can depend on the clinician to align and advance a terminal end of the guidewire to make contact with an optical contact within the interface. Also, user turnable actuators can be difficult or inconvenient for the clinician to actuate during surgery. The present inventors have recognized among other things, devices and methods enabling recurrent convenient and reliable connections between an interface and a terminal end of the guidewire during a procedure.

This document describes, among other things, devices and methods enabling recurrent convenient and reliable connections between an interface and a terminal end of the guidewire during a procedure. Devices described herein can include or use a dual-action mechanism enabling concurrent actuation of a gripping mechanism and a guidewire-advancing mechanism. This can help provide ergonomic, accurate, and fast connection of the guidewire, including helping orient and optically couple one or more optical fibers during a procedure. The guidewire can be easy for a physician to disconnect or re-connect such as at a location within the sterile field, which can be wet and bloody. The interface can include or use a flush port such as to mitigate contamination at or near optical contacts by elements from the sterile field. Devices herein can also include or use a stabilization cradle such as to help restrict linear movement of the interface during the procedure while permitting rotational movement of the interface.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 depicts an example of an optical hemodynamics system.

FIG. 2 depicts an example of an optical hemodynamics system in use with a patient, such as for hemodynamic sensing.

FIG. 3A depicts an example of an optical system lead connector.

FIG. 3B depicts an example of an optical system lead connector.

FIG. 4A depicts an example of an optical interface of a system lead connector.

FIG. 4B depicts a cross sectional view of an example of a pressure guidewire taken along the cut-line shown in FIG. 4A.

FIG. 5A depicts an example of an optical interface of a system lead connector.

FIG. 5B depicts a magnified view of an example of a cam and follower feature of the optical interface of a system lead connector shown in FIG. 5A.

FIG. 5C depicts a cross sectional view of an example of a pressure guidewire taken along the cut-line shown in FIG. 5A.

FIG. 6A depicts a cross section of the example of an optical interface of a system lead connector shown in FIG. 5A.

FIG. 6B depicts a cross section of the example of an optical interface of a system lead connector shown in FIG. 5A.

FIG. 6C depicts a cross section of the example of an optical interface of a system lead connector shown in FIG. 6A.

FIG. 6D depicts a cross section of the example of an optical interface of a system lead connector shown in FIG. 6B.

FIG. 7A depicts a plug of an example of a system lead connector.

FIG. 7B depicts a cross sectional view of an example of the plug of an example of a system lead connector taken along the cut-line shown in FIG. 7A.

FIG. 8A depicts an example of a stabilization cradle connecting to an optical interface of a system lead connector.

FIG. 8B depicts an example of a stabilization cradle connecting to an optical interface of a system lead connector.

FIG. 9 is an example of a flowchart that depicts a method of using a system lead connector device.

### DETAILED DESCRIPTION

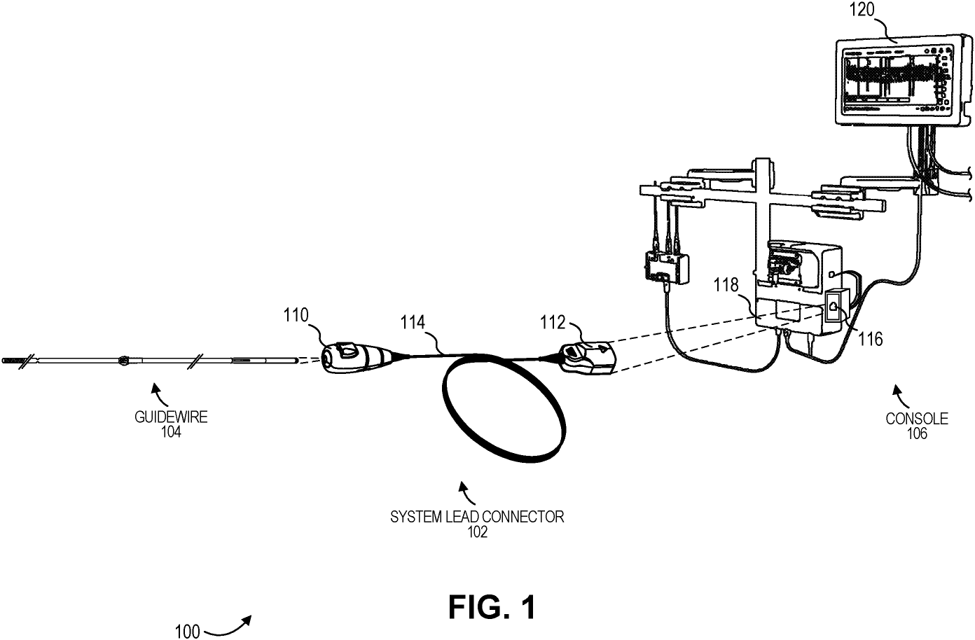

This document describes, among other things, pressure sensing devices, imaging devices, and methods using optical elements and techniques. For example, a pressure guidewire can be used with an optical hemodynamics system such as to help measure and analyze hemodynamics during a procedure. FIG. 1 depicts an example of an optical hemodynamics system 100. The optical hemodynamics system can include or use a system lead connector 102 for operably coupling a pressure guidewire 104 to a console 106. For example, the guidewire can be inserted at an optical interface 110 of the connector 102. The interface 110 can operably couple an optical fiber contained within the pressure guidewire 104 to an interface cable 114. The interface cable 114 can be terminated, e.g., with a plug 112 such as a fiber optic (FC) connector. The plug 112 can be sized and shaped such as to couple with a system lead receptacle 116 of the console 106. For example, the system lead receptacle 116 can be operably connected to an acquisition engine (AE) 118 of the console. The AE 118 can receive data collected from a distal end of the guidewire 104 during a procedure, and the data can be analyzed and feedback can be provided to a user, such as via a monitor 120.

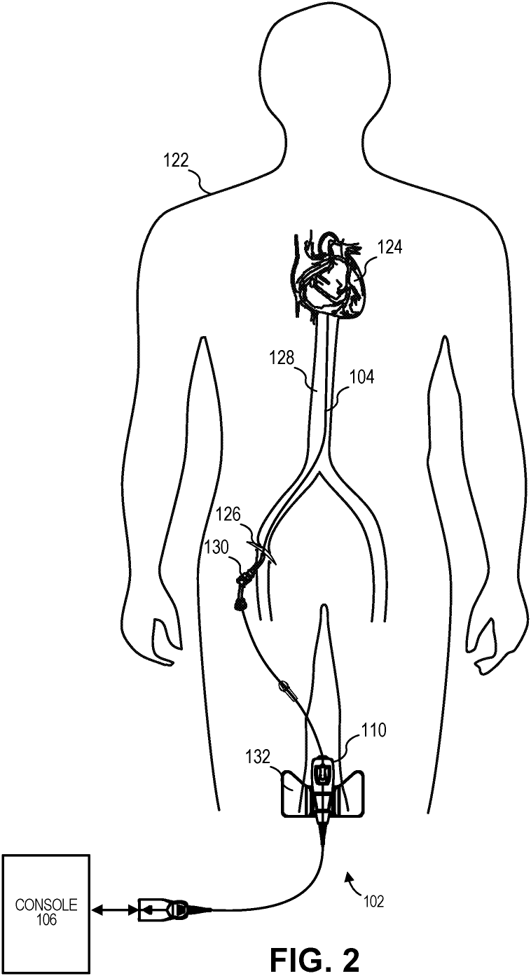

FIG. 2 depicts an example of an optical hemodynamics system in use with a patient such as for hemodynamic sensing. For example, a patient 122 can be positioned lying supine, such as on an operating table. An incision 126 can be created or used such as to provide the clinician with access to a vessel 128. A catheter 130 can be included or used such as to provide a conduit through the incision 126 and into the vessel 128. The pressure guidewire 104 can be inserted through the catheter 130, e.g., into the catheter, and the guidewire 104 can be advanced through the vessel 128. The distal end of the guidewire 104 can be advanced to a location at or near the heart 124 or the patient. The proximal end of the guidewire 104 can be operably connected with the optical interface 110 of the system lead connector 102, and the system lead connector can relay pressure measurement data collected at or near the distal end of the guidewire 104 to the console 106.

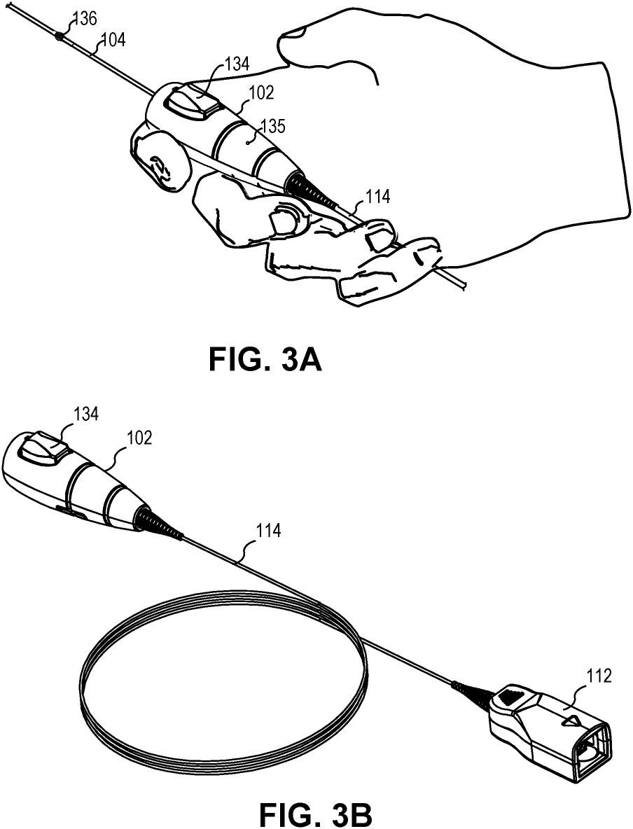

FIG. 3A and FIG. 3B depict an example of an optical system lead connector. The optical interface 110 can include or use an actuator 134 and a torquer 136. In an example, the proximal end of the guidewire 104 can be fed into an aperture of the interface 110 at or near the torquer 136. The actuator 134 can be operated such as such as to help clamp or release the guidewire from the interface 110. The torquer 136 can be manipulated by the clinician, such as between the thumb and index finger, to help manipulate the guidewire 104 during the procedure.

In an example, the interface 110 can rotate dependent on rotation caused by turning of the torquer 136. For example, the interface 110 cannot be rotatable independent of the torquer. Synchronous or corresponding rotation of the torquer 136 and the interface 110 can help preserve the integrity of an optical connection between the proximal end of the guidewire 104 and the interface. Alternatively or additionally, the interface 110 can be rotatable independent of the torquer.

As depicted in FIG. 3B, the interface 110 can be operably connected to the plug 112 via the interface cable 114. In an example, the interface cable can include or use single mode optical fiber including a core having a diameter of less than 10.5 micrometers (µm). For example, the single mode optical fiber can include a core with a diameter between about 8 µm and about 10.5 µm.

In an example, the optical interface 110 can include or use an integrated cleaning mechanism such as a flush port 135. The cleaning mechanism can be used to remove debris from the ends of the guidewire assembly before the guidewire is coupled together within the interface 110. Debris, such as blood and other bodily fluids, trapped between the proximal end of the guidewire 104 or the receptacle of the interface can reduce the performance of the optical guidewire. In a surgical environment, reduced performance of the optical guidewire can create unnecessary delay in treating a patient. In an example, the flush port 135 can be included within the housing of the interface. The flush port 135 can be a channel arranged such as to deliver solution inserted at the port to the optical receptacle, the proximal end of the guidewire, or other elements of the optical interface 110. Alternatively or additionally, the optical interface 110 can include or use a flush port 135 located at or near the aperture. A saline solution can be administered to the flush port 135, such as by a syringe, and the solution can be withdrawn, bled, or otherwise removed from the optical interface 110 out of an ejection port of the housing.

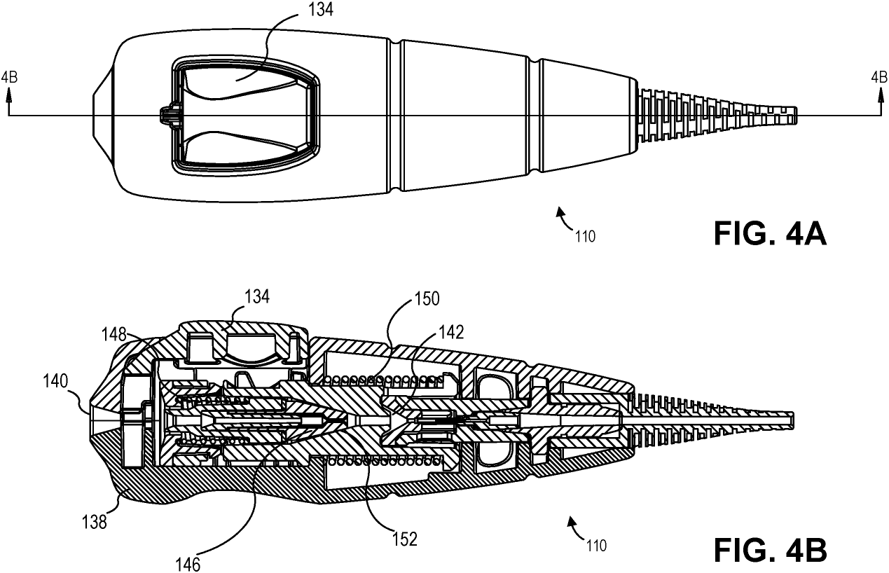

FIG. 4A and FIG. 4B depict an example of an optical interface of a system lead connector. In an example, the optical interface 110 can include or use an actuator 134, housing 138 defining an aperture 140, an optical receptacle 142 disposed within the housing, and a chuck 144. The actuator 134 can be a trigger, button, a switch, a knob, a lever, or other type of actuator ergonomically available to the clinician during a procedure. For example, the actuator 134 can be a trigger arranged such as to permit dual-action actuation. The actuator 134 can be operated between first trigger position wherein the trigger is biased by a trigger bias in a lateral direction from the housing 138 and a second trigger position wherein the trigger is depressed medially towards the housing 138 and against the trigger bias. The trigger bias can apply force to the actuator 134 such that the clinician can easily actuate the trigger with one finger while mitigating accidental actuation and ejection of the guidewire from the optical interface 110. In an example, a spring force of the trigger bias can be within a range of about 0.5 pound-force (lbf) to about 1.2 lbf.

The dual-action actuation of the actuator 134 can include or use tightening/loosening the chuck 144 concurrent with advancing/retracting the chuck in a lateral direction towards the optical receptacle 142. For example, a chuck can clamp around the guidewire extending through the aperture 140. The chuck 144 can be slidable within the housing between a first chuck position wherein the chuck is positioned closer to the optical receptacle than to the aperture and a second chuck position wherein the chuck is positioned closer to the aperture than to the optical receptacle. A singular operation of the actuator 134, such as, e.g., depression or release thereof, can laterally move the chuck between the first position and the second position and concurrently tighten or loosen the chuck.

FIG. 4B depicts a cross sectional view of an example of an optical interface taken along the cut-line shown in FIG. 4A. In an example, the optical interface 110 can also include or use a chuck bias 148 and an optical connection bias 148. The chuck bias 148 can be a spring mechanism and the bias 148 can help dictate the amount of force used to advance the chuck to the first position. In an example, the chuck 146 can be a collet sized and shaped to interface with a taper 152. Effectively, lateral movement between the first and second chuck positions can dictate how hard collet jaws or the collet clamp down onto guidewire extending through the collet since the lateral movement towards the first chuck position pushes the collet into the taper. In an example, a spring force of the chuck bias 148 can be within a range of about 1.5 pound-force (lbf) to about 10 lbf.

The optical connection bias 150 can be spring mechanism and can help dictate the amount of force used to push the proximal end of the guidewire 104 together with the optical receptacle 142. This force can be critical to help ensure a good optical connection. Too little force applied by the optical connection bias 150 can allow a lack of engagement or contact between the proximal end of the guidewire 104 and the optical receptacle 142. Too much force applied by the optical connection bias 150 can promote buckling and can prevent proper alignment. In an example, a spring force of the optical connection bias 150 can be within a range of about 0.40 lbf to about 0.50 lbf.

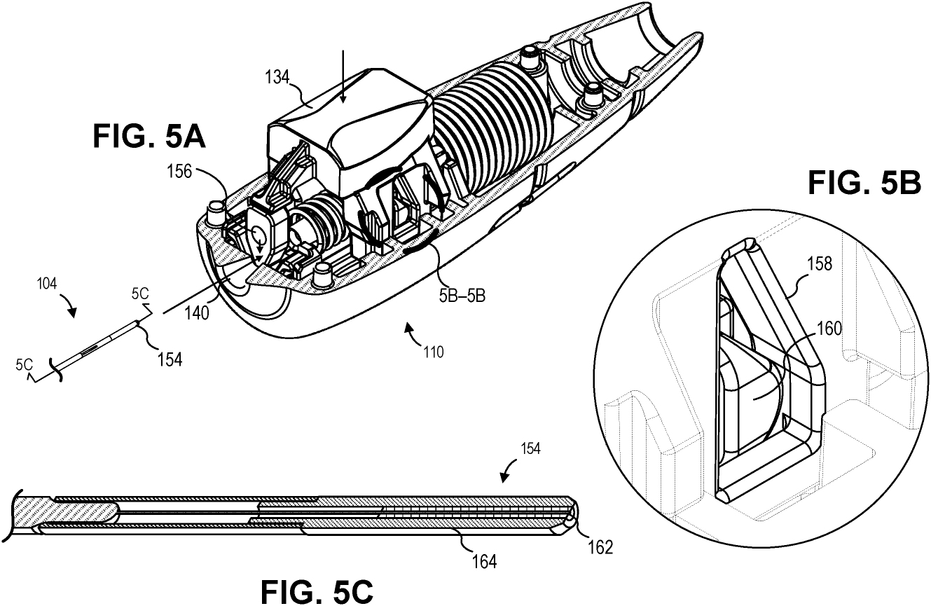

FIG. 5A depicts an example of an optical interface of a system lead connector. As illustrated in FIG. 5A, a proximal end 154 of the guidewire 104 can be inserted at the aperture 140 optical interface 110. In an example, the optical interface 110 can include or use a window 156. The window 156 can include or use a window bias such that when the window is in a biased position, advancement of the proximal end 154 of the guidewire 104 into the optical interface 110 is impeded. The window 156 can be retracted such as to allow for entry of the guidewire 104. For example, operation of the actuator 134 can retract the window 156 against the window bias concurrent with actuating other mechanisms described herein.

In an example, the actuator 134 can be depressed, concurrently loosening the chuck and retracting or opening the window 156 such as to allow for the proximal end 154 of the guidewire 104 to be advanced through the aperture 140 of the optical interface 110. Also, after the guidewire 104 has been advanced into the chuck 146, movement of the window 156 back towards the biased position upon release of the actuator 134 can help the window 156 clamp on the guidewire 104 and provide supplemental gripping thereof.

FIG. 5B depicts a magnified view of an example of a cam and follower feature of the optical interface of a system lead connector shown in FIG. 5A. In an example, the actuator 134 can include or use or be operable connected to a cam feature 158. The cam feature can be arranged such that when the actuator 134 is operated, the cam feature 158 contacts a follower 160 included, used by, or operably connected to the chuck such as to manipulate lateral movement of the chuck. Operation of the actuator 134 to contact the cam feature 158 with the follower 160 can help move the chuck 146 against the chuck bias 148 (depicted in FIG. 4B).

In an example, the optical interface 110 can include or use or be operably coupled to a plurality of cam features 158 arranged such as to contact a corresponding follower 160 of a plurality of followers 160 of the chuck. For example, two cam-follower pairs can be included in the optical interface 110 such as to help maintain alignment while moving the chuck against the chuck bias during operation of the actuator 134.

FIG. 5C depicts a cross sectional view of an example of a pressure guidewire taken along the cut-line shown in FIG. 5A. Here, optical fiber 162 can be encapsulated along the length of the assembly such as in a protective coating, such as a plastic matrix at the proximal end 154 of the guidewire 104. The guidewire 104 can include or use an internal optical fiber that carries the light signal to the distal end of the guidewire 104 and back to console 106. Here, both optical fiber in the guidewire 104 and the optical fiber in the system lead connector 102 must to be coaxially aligned and held in contact during use.

In an example, the optical fiber can be a single mode optical fiber including a core having a diameter of less than 10.5 µm. For example, the single mode optical fiber can include a core with a diameter between about 8 µm and about 10.5 µm. A small misalignment between optical fiber cores can produce significant optical coupling loss. In an example, the optical fiber 162 can be encapsulated within a split sleeve 164. As discussed below, the split sleeve 164 can include a taper such as to help align the optical fiber 162 with an optical contact in the optical receptacle of the optical interface 110.

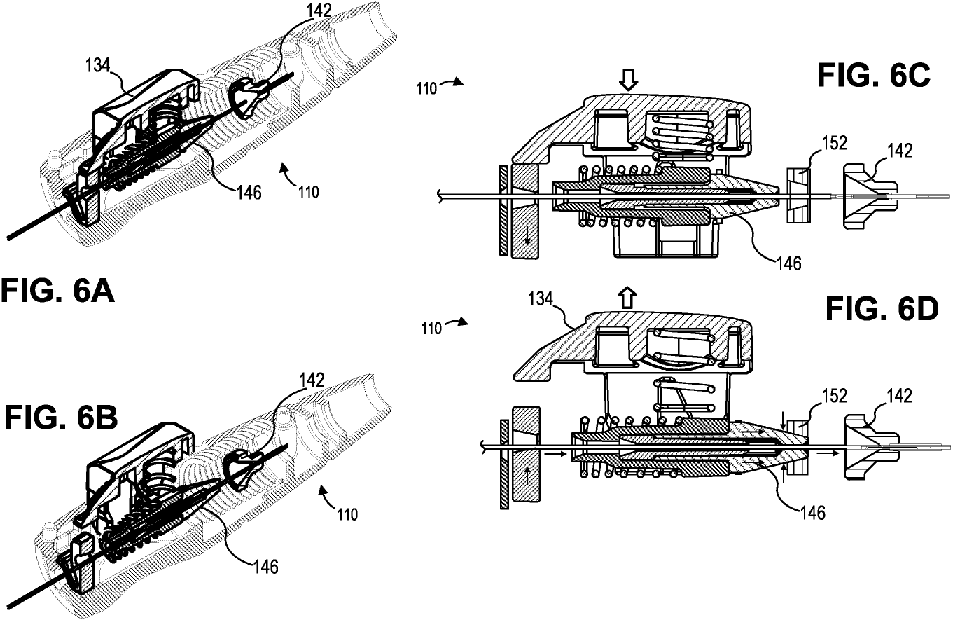

FIGS. 6A-6D depict a cross section of the example of an optical interface of a system lead connector shown in FIG. 5A. As depicted, the chuck 146 can be moved between the second chuck position (shown in FIG. 6A; FIG. 6C) and the first chuck position (shown in FIG. 6B; FIG. 6D) by a single operation of the actuator 134 such as depression of a trigger or button. Also, actuating the chuck 146 to move to the second chuck position can loosen the chuck 146, and actuating the chuck to move to the first chuck position can tighten the chuck 146. For example, operating the actuator 134 to move the chuck 146 to the first chuck position can concurrently increase an inner diameter defined by jaws of the chuck 146.

Also, operating the actuator 134 to move the chuck to the second chuck position can concurrently decrease the inner diameter defined by jaws of the chuck 146. For example, the when the actuator 134 is moved to the first trigger position, the chuck can be moved to the first chuck position and an inner diameter defined by jaws of the chuck can concurrently be increased. Also, when the actuator 134 is moved to the second trigger position, the chuck can be moved to the second chuck position and the inner diameter defined by jaws of the chuck can concurrently be decreased.

In an example, the concurrent mechanical actuation of both the lateral movement of the chuck 146 as well as tightening or loosening of the chuck can result a timing offset of these actions from each other. For instance, it can be desirable that operation of the actuator 134 first tightens or loosens the chuck 146 and slightly delays lateral movement of the chuck. This can help ensure that the guidewire 104 is properly gripped or released before being manipulated by the chuck 146 in the lateral position. For example, as previously described with respect to FIG. 4B the chuck 146 can be a collet arranged within a taper 152. Operation of the actuator 134 can release a counterforce the chuck bias 148 (such as force provided by the cam-follower mechanism as depicted in FIG. 5B) before releasing a counterforce against the connection bias 150 (depicted in FIG. 4B). As such, the collet can be advanced within the taper 152, tightening the jaws of the collet, before the connection bias 150 (depicted in FIG. 4B) is allowed to manipulate the collet towards the optical receptacle 142.

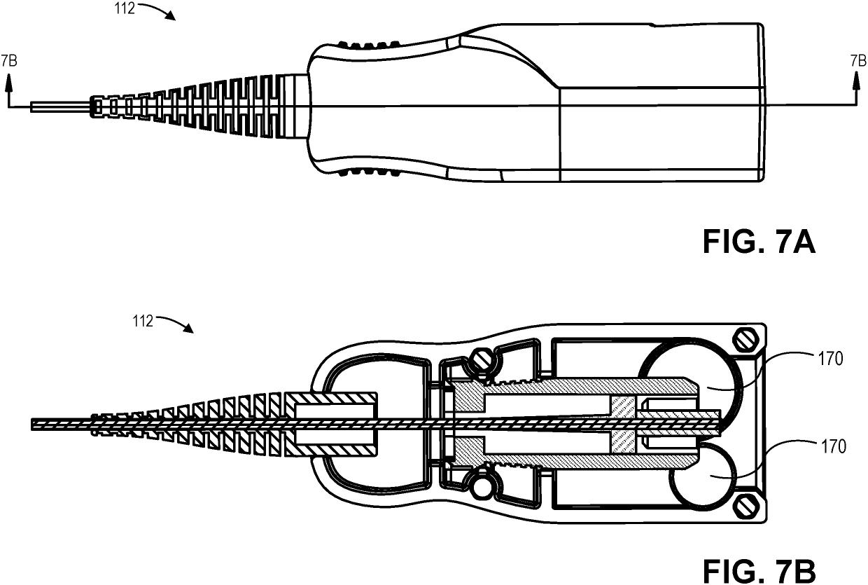

FIG. 7A depicts a plug of an example of a system lead connector. In an example, the plug 112 can be a lucent connector (LC), fiber-optic connector (FC), a push pull connector, small form factor (SFF) connector, field assembly connector, ferrule connector, fiber channel, or other type of suitable fiber-optic connector. Examples of fiber-optic polishes included in the plug 112 can include FC/PC (physical contact), FC/APC (angled physical contact).

FIG. 7B depicts a cross sectional view of an example of the plug of an example of a system lead connector taken along the cut-line shown in FIG. 7A. In an example, the plug 112 can include or use one or more console-identifiable features 170 embedded within the plug 112. For example, the console-identifiable feature 170 can be a magnet. Upon inserting the plug into the system lead receptacle 116 (as depicted in FIG. 1), the console can recognize a magnetic or resonant signal from the magnet such as to identify the presence of a system lead connector. Alternatively or additionally, the console-identifiable feature can be a compliance indicator. The compliance indicator can also include or use a radio-frequency identification (RFID) device. For example, the compliance indicator can include or use an initiator such as an near field communication (NFC) chip attached to or embedded within the feature 170 for communication with, e.g., the communications circuitry of the console. The compliance indicator can transmit, e.g., a radiofrequency signal to be read by an indicator reader included in communications circuitry. The communications circuitry can include or use memory including instructions to impede one or more system functions upon detection (or instructions of) system lead connector without a compliant indicator. The RFID device can be a radiation-hardened device such as to mitigate erasure or damage of the device during sanitation of the system lead connector. For instance, the radiation-hardened RFID device can withstand electron beam (e-beam) sanitation without losing console identification capabilities.

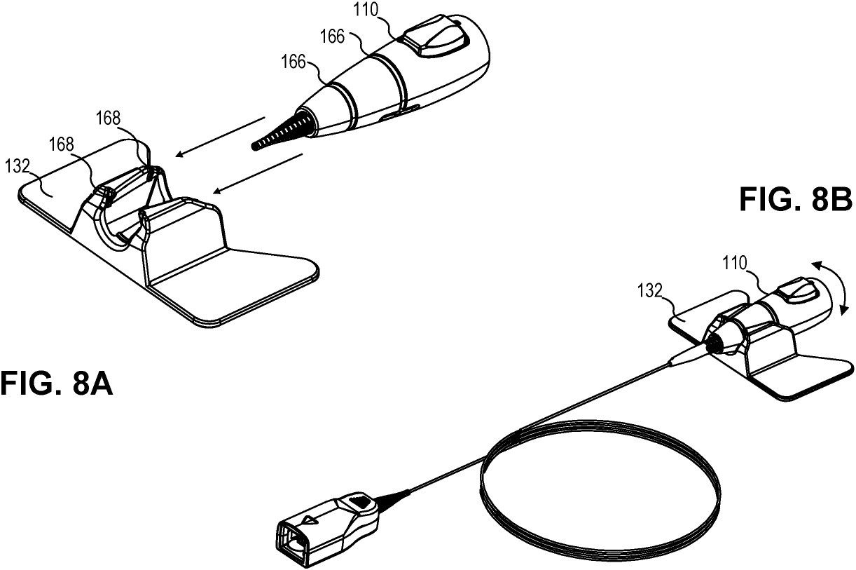

FIG. 8A and FIG. 8B depict an example of a stabilization cradle connecting to an optical interface of a system lead connector. In an example, the optical interface 110 can include or use one or more interface mating features 166 for connecting with one or more corresponding cradle mating features 168 of the stabilization cradle 132. For example, the mating features can be tongues sized and shapes to ride corresponding mating features which are grooves. For instance, at least one cradle mating feature 166 or at least one optical interface mating feature can be a radial groove arranged around an axis between the aperture of the optical interface 110 and the optical receptacle thereof.

The stabilization cradle 132 can be couplable to the optical interface 110 and configured to restrict linear movement of the optical interface 110 during the procedure while permitting rotational movement of the optical interface 110 during the procedure. For example, the optical interface 110 can be a handle or a shaft for manipulating the guidewire. In an example, the optical interface 110 can be manipulated by the clinician by turning the torquer 136 operably connected to the optical interface 110. The optical interface 110 can be easily and ergonomically coupled and uncoupled from the stabilization cradle 132 during the procedure by advancing or snapping the optical interface 110 laterally into the cradle 132 while restricting uncoupling by pulling the optical interface 110 vertically away from the cradle.

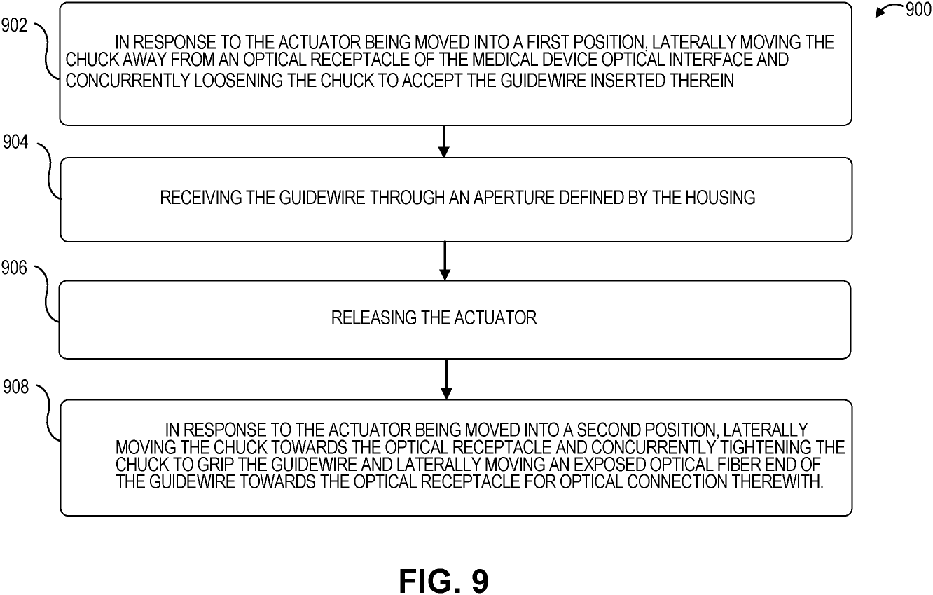

FIG. 9 is an example of a flowchart that depicts a method of using a system lead connector device. At 902, in response to the actuator being moved into a first position, the chuck can be laterally moved away from an optical receptacle of the medical device optical interface and the chuck can be concurrently loosened such as to accept the guidewire inserted therein. For example, the actuator can be a trigger or button. For example, the trigger can be biased in a lateral direction from the housing when the trigger is in the first trigger position.

At 904, the guidewire can be received through an aperture defined by the housing. At 906, the actuator can be released. At 908, in response to the actuator being moved into a second position, the chuck can be laterally moved towards the optical receptacle and the chuck can be concurrently tightened such as to grip the guidewire and an exposed optical fiber end of the guidewire can be moved laterally towards the optical receptacle for optical connection therewith. For example, in response to the actuator being moved into the first position, the chuck can be moved to a first chuck position and an inner diameter defined by jaws of the chuck can be concurrently increased. Also, in response to the actuator being moved into a second position, the chuck can be moved to a second chuck position and the inner diameter defined by jaws of the chuck can be concurrently decreased. Also, where the chuck includes a collet arranged within a taper inside the housing, the jaws of the chuck can be compressed towards one another when the collet is moved from the second chuck position to the first chuck position. The optical receptacle can be communicatively coupled to an optical signal analysis console for transmitting an optical signal from the optical fiber to the optical signal analysis console. Saline solution can be received or administered such as at a flush port of the medical device optical interface to the optical receptacle.

Example 1 is a medical device optical connector for manipulating a guidewire including optical fiber, the connector comprising: a housing defining an aperture; an optical receptacle disposed within the housing, the optical receptacle configured to receive an exposed optical fiber end of the guidewire extending through the aperture; a chuck configured to clamp around the guidewire extending through the aperture, wherein the chuck is slidable within the housing between: a first chuck position wherein the chuck is positioned closer to the optical receptacle than to the aperture; and a second chuck position wherein the chuck is positioned closer to the aperture than to the optical receptacle; and an actuator configured to laterally move the chuck between the first position and the second position and concurrently tighten or loosen the chuck.

In Example 2, the subject matter of Example 1, wherein the actuator is configured to: move the chuck to the first chuck position and concurrently increase an inner diameter defined by jaws of the chuck; and move the chuck to the second chuck position and concurrently decrease the inner diameter defined by jaws of the chuck.

In Example 3, the subject matter of Example 2, wherein the actuator is a trigger configured to move between: a first trigger position wherein the trigger is biased in a lateral direction from the housing; and a second trigger position wherein the trigger is depressed medially towards the housing and against the bias.

In Example 4, the subject matter of Example 3, wherein: the first trigger position moves the chuck to the first chuck position and concurrently increase an inner diameter defined by jaws of the chuck; and the second trigger position moves the chuck to the second chuck position and concurrently decrease the inner diameter defined by jaws of the chuck.

In Example 5, the subject matter of any of Examples 1-4, wherein: the chuck includes a collet arranged within a taper inside the housing; and jaws of the collet are configured to compress towards one another when the collet is moved from second chuck position to the first chuck position.

In Example 6, the subject matter of any of Examples 1-5, wherein the optical receptacle is communicatively couplable to an optical signal analysis console for transmitting an optical signal from the optical fiber to the optical signal analysis console.

In Example 7, the subject matter of Example 6, wherein the optical fiber is a single mode optical fiber (SMF), and wherein the optical receptacle is configured to communicatively couple with the single mode optical fiber (SMF) to the optical signal analysis console.

Incorrect Numbering : 8

In Example 8, the subject matter of any of Examples 6-7, wherein the optical receptacle is configured to communicatively couple with the optical fiber and to communicatively connect the optical fiber to the optical signal analysis console, wherein the optical fiber includes a core with a diameter between about 8 µm and about 8 is missing parent: 10.5 µm.

In Example 9, the subject matter of any of Examples 1-8, wherein the actuator configured to laterally move the chuck between the first position and the second position, concurrently tighten or loosen the chuck, and concurrently open or close a window of the aperture.

In Example 10, the subject matter of any of Examples 1-9, wherein the housing comprises a flush port configured to deliver solution inserted at the port to the optical receptacle.

Example 11 is a method for attaching a guidewire including an optical fiber to an optical signal analysis console using a medical device optical interface, wherein the interface is couplable to the optical signal analysis console, and wherein the connector includes a housing, a trigger coupled to a housing, and a chuck configured to grip the guidewire extending through an aperture defined by the housing, the method comprising: in response to the trigger being moved into a first trigger position, laterally moving the chuck away from an optical receptacle of the medical device optical interface and concurrently loosening the chuck to accept the guidewire inserted therein; receiving the guidewire through an aperture defined by the housing; releasing the trigger; and in response to the trigger being moved into a second trigger position, laterally moving the chuck towards the optical receptacle and concurrently tightening the chuck to grip the guidewire and laterally moving an exposed optical fiber end of the guidewire towards the optical receptacle for optical connection therewith.

In Example 12, the subject matter of Example 11, comprising: biasing the trigger in a lateral direction from the housing when the trigger is in the first trigger position.

In Example 13, the subject matter of any of Examples 11-12, wherein in response to the trigger being moved into a first trigger position includes: moving the chuck to a first chuck position and concurrently increasing an inner diameter defined by jaws of the chuck; and wherein in response to the trigger being moved into a second trigger position includes: moving the chuck to the second chuck position and concurrently decreasing the inner diameter defined by jaws of the chuck.

In Example 14, the subject matter of Example 13, wherein the chuck includes a collet arranged within a taper inside the housing, the method comprising: compressing the jaws of the chuck towards one another when the collet is moved from second chuck position to the first chuck position.

In Example 15, the subject matter of any of Examples 11-14, comprising: communicatively coupling the optical receptacle to the optical signal analysis console for transmitting an optical signal from the optical fiber to the optical signal analysis console.

In Example 16, the subject matter of any of Examples 11-15, wherein the optical fiber is a single mode optical fiber (SMF), the method comprising: communicatively coupling the single mode optical fiber (SMF) to the optical signal analysis console.

In Example 17, the subject matter of any of Examples 11-16, comprising: communicatively coupling the optical fiber with the optical receptacle and to the optical signal analysis console, wherein optical fiber includes a core with a diameter between about 8 µm and about 10.5 µm.

In Example 18, the subject matter of any of Examples 11-17, comprising: receiving saline solution at a flush port of the medical device optical interface to the optical receptacle.

Example 19 is a medical system for surgical optical analysis during a surgical procedure using a guidewire including an optical fiber, the system comprising: an optical signal analysis console for receiving an optical signal and configured to represent a visual image corresponding to the optical signal; and a system lead connector comprising: a first end including an optical connector for coupling the system lead connector with the optical signal analysis console; a second end including a handle comprising: a housing defining an aperture; an optical receptacle disposed within the housing, the optical receptacle configured to receive an exposed optical fiber end of the guidewire extending through the aperture; a chuck configured to clamp around the optical fiber extending through the aperture, wherein the chuck is slidable within the housing between: a first chuck position wherein the chuck is positioned closer to the optical receptacle than to the aperture; and a second chuck position wherein the chuck is positioned closer to the aperture than to the optical receptacle; and an actuator configured to laterally move the chuck between the first position and the second position and concurrently tighten or loosen the chuck; and an optical connection between the optical connector and the handle; and a connector stabilization cradle couplable to the handle and configured to restrict linear movement of the handle during the procedure while permitting rotational movement of the handle during the procedure.

In Example 20, the subject matter of Example 19, wherein the stabilization cradle includes at least one cradle mating feature sized and shaped to conform to at least one handle mating feature on the housing of the handle.

In Example 21, the subject matter of any of Examples 19-20, wherein the at least stabilization cradle mating feature or the at least one handle mating feature is a radial groove arranged around an axis between the aperture and the optical receptacle.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

Each of these non-limiting examples can stand on its own, or can be combined in various permutations or combinations with one or more of the other examples.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical device optical connector lead for coupling with a guidewire including optical fiber, the connector comprising:
a housing defining an aperture;
an optical receptacle disposed within the housing, the optical receptacle configured to receive an exposed optical fiber end of the guidewire extending through the aperture;
a chuck configured to clamp around the guidewire extending through the aperture, wherein the chuck is slidable within the housing between:
a first chuck position wherein the chuck is positioned closer to the optical receptacle than to the aperture; and
a second chuck position wherein the chuck is positioned closer to the aperture than to the optical receptacle; and
an actuator configured to laterally move the chuck between the first position and the second position and concurrently tighten or loosen the chuck.

2. The connector of claim 1, wherein the actuator is configured to:
move the chuck to the first chuck position and concurrently increase an inner diameter defined by jaws of the chuck; and
move the chuck to the second chuck position and concurrently decrease the inner diameter defined by jaws of the chuck.

3. The connector of claim 2, wherein the actuator is a trigger configured to move between:
a first trigger position wherein the trigger is biased in a lateral direction from the housing; and
a second trigger position wherein the trigger is depressed medially towards the housing and against the bias.

4. The connector of claim 3, wherein:
the first trigger position moves the chuck to the first chuck position and concurrently increase an inner diameter defined by jaws of the chuck; and
the second trigger position moves the chuck to the second chuck position and concurrently decrease the inner diameter defined by jaws of the chuck.

5. The connector of claim 1, wherein:
the chuck includes a collet arranged within a taper inside the housing; and
jaws of the collet are configured to compress towards one another when the collet is moved from second chuck position to the first chuck position.

6. The connector of claim 1, wherein the optical receptacle is communicatively couplable to an optical signal analysis console for transmitting an optical signal from the optical fiber to the optical signal analysis console.

7. The connector of claim 6, wherein the optical fiber is a single mode optical fiber (SMF), and wherein the optical receptacle is configured to communicatively couple with the single mode optical fiber (SMF) to the optical signal analysis console.

8. The connector of claim 6, wherein the optical receptacle is configured to communicatively couple with the optical fiber and to communicatively connect the optical fiber to the optical signal analysis console, wherein the optical fiber includes a core with a diameter between about 8 µm and about 10.5 µm.

9. The connector of claim 1, wherein the actuator configured to laterally move the chuck between the first position and the second position, concurrently tighten or loosen the chuck, and concurrently open or close a window of the aperture.

10. The connector of claim 1, wherein the housing comprises a flush port configured to deliver solution inserted at the port to the optical receptacle.

11. A method for attaching a guidewire including an optical fiber to an optical signal analysis console using a medical device optical interface, wherein the interface is couplable to the optical signal analysis console, and wherein the connector includes a housing, a trigger coupled to a housing, and a chuck configured to grip the guidewire extending through an aperture defined by the housing, the method comprising:
in response to the trigger being moved into a first trigger position, laterally moving the chuck away from an optical receptacle of the medical device optical interface and concurrently loosening the chuck to accept the guidewire inserted therein;
receiving the guidewire through an aperture defined by the housing;
releasing the trigger; and
in response to the trigger being moved into a second trigger position, laterally moving the chuck towards the optical receptacle and concurrently tightening the chuck to grip the guidewire and laterally moving an exposed optical fiber end of the guidewire towards the optical receptacle for optical connection therewith.

12. The method of claim 11, comprising:
biasing the trigger in a lateral direction from the housing when the trigger is in the first trigger position.

13. The method of claim 11,
wherein in response to the trigger being moved into the first trigger position includes:
moving the chuck to a first chuck position and concurrently increasing an inner diameter defined by jaws of the chuck; and
wherein in response to the trigger being moved into the second trigger position includes:
moving the chuck to a second chuck position and concurrently decreasing the inner diameter defined by jaws of the chuck.

14. The method of claim 13, wherein the chuck includes a collet arranged within a taper inside the housing, the method comprising:
compressing the jaws of the chuck towards one another when the collet is moved from the second chuck position to the first chuck position.

15. The method of claim 11, comprising:
communicatively coupling the optical receptacle to the optical signal analysis console for transmitting an optical signal from the optical fiber to the optical signal analysis console.

16. The method of claim 11, wherein the optical fiber is a single mode optical fiber (SMF), the method comprising:
communicatively coupling the single mode optical fiber (SMF) to the optical signal analysis console.

17. The method of claim 11, comprising:
communicatively coupling the optical fiber with the optical receptacle and to the optical signal analysis console, wherein optical fiber includes a core with a diameter between about 8 µm and about 10.5 µm.

18. The method of claim 11, comprising:
receiving saline solution at a flush port of the medical device optical interface to the optical receptacle.

19. A medical system for surgical optical analysis during a surgical procedure using a guidewire including an optical fiber, the system comprising:
an optical signal analysis console for receiving an optical signal and configured to represent a visual image corresponding to the optical signal; and
a system lead connector comprising:
a first end including an optical connector for coupling the system lead connector with the optical signal analysis console;
a second end including a handle comprising:
a housing defining an aperture;
an optical receptacle disposed within the housing, the optical receptacle configured to receive an exposed optical fiber end of the guidewire extending through the aperture;
a chuck configured to clamp around the optical fiber extending through the aperture, wherein the chuck is slidable within the housing between:
a first chuck position wherein the chuck is positioned closer to the optical receptacle than to the aperture; and
a second chuck position wherein the chuck is positioned closer to
the aperture than to the optical receptacle; and
an actuator configured to laterally move the chuck between the first position and the second position and concurrently tighten or loosen the chuck; and
an optical connection between the optical connector and the handle; and
a connector stabilization cradle couplable to the handle and configured to restrict linear movement of the handle during the procedure while permitting rotational movement of the handle during the procedure.

20. The system of claim 19, wherein the stabilization cradle includes at least one cradle mating feature sized and shaped to conform to at least one handle mating feature on the housing of the handle.

21. The system of claim 19, wherein the at least one cradle mating feature or the at least one handle mating feature is a radial groove arranged around an axis between the aperture and the optical receptacle.
